(19) United States
(12) Patent Application Publication
(10) Pub. No.: US 2024/0392250 A1
(43) Pub. Date: Nov. 28, 2024

(54) HIGH-THROUGHPUT SELECTION AND FABRICATION OF BIOMATERIAL-ENCAPSULATED CELL MASS AND USES THEREOF

(71) Applicant: BioArchitec Group Limited, Hong Kong (CN)

(72) Inventors: Chung Wai Jonathan LAM, Hong Kong (CN); Chi Hang WONG, Hong Kong (CN); Chi Hang WONG, Hong Kong (CN); Chiu Chin PONG, Hong Kong (CN)

(21) Appl. No.: 18/260,835

(22) PCT Filed: May 19, 2023
(86) PCT No.: PCT/CN2023/095216

§ 371 Date: Jul. 10, 2023

Related U.S. Application Data
(60) Provisional application No. 63/365,204, filed on May 24, 2022.
(60) Provisional application No. 63/369,256, filed on Jul. 25, 2022.

(51) Int. Cl. C12N 5/09; C12N 5/00

(52) U.S. Cl. CPC C12N 5/0693; C12N 5/0012; C12N 2503/02; C12N 2513/00; C12N 2533/30; C12N 2533/54; C12N 2537/10

(57) ABSTRACT

The present invention provides a method for high-throughput selection of cell masses from a source and fabrication of biomaterial-encapsulated cell mass model based on the selected cell masses, the as-fabricated cell mass model or cells derived therefrom, their applications in various areas including both in vivo and in vitro studies, and potentials to develop into different therapies.

### CROSS-REFERENCE TO RELATED APPLICATION

This application is a 371 application of International Patent Application under the application number PCT/CN2023/095216 filed May 19, 2023 which claims priorities from (1) the U.S. provisional patent application Ser. No. 63/365,204 filed May 24, 2022; and (2) the U.S. provisional patent application Ser. No. 63/369,256 filed Jul. 25, 2022, the disclosures of which are incorporated herein by reference in their entirety.

### TECHNICAL FIELD

The present invention relates to a method for high-throughput selection and fabrication of biomaterial-encapsulated cell mass (BECM), and uses of the BECM.

### BACKGROUND

Cell masses, including single-cell type spheroids and multi-cell type organoids derived from any living organisms, are important models for in vitro and in vivo studies such as cancer research, drug screening, and drug sensitivity test for precision medicine. However, current technologies result in inconsistent cell number and sizes during the production of these cell masses. In the context of cancer research, the composition of normal and cancerous content in patient-derived organoids (PDO) is highly inconsistent among individual cell masses. The tissue source for PDO culturing taken from the patient is usually limited in size, which also limits the number of organoid cell masses that can be produced, and therefore the subsequent tests that can be performed. This hinders their applications in drug test and development with the recent advancement in precision medicine.

Due to the inherent tumor cell diversity, researchers usually derive cell masses from primary cells after careful selection, but current technologies hinder their selective power and control over the size, composition, and cell number of the cell masses such as spheroids and organoids derived from tissues or biopsy of a donor. In case of multi-cell type organoids, a mixture of highly variable compositions and sizes of the cell masses inherently imposes inconsistency to experimental results, which are usually compensated by increasing the sample size. To allow subsequent tests, the multi-cell type organoids should be digested into single cells before seeding equal number of cells in each well for accurate comparisons. This involves an assumption that all the cell compositions are equally divided and the multi-cell type organoids grown from the single cells are formed at the same rate with similar compositions. However, each individual cell mass is in fact different from the other, so the assumption as such leads to an even more inconsistent result.

Cell sorting by flow cytometry or cell sorter could be a solution to attempt to standardize the cell size or cell stage by biomarkers, but it involves time and labors, and treatment of the cells before sorting may cause other unexpected result to the cell fate and its ability to grow into a desired biological model or cell lineage for various tests.

A need therefore exists for an improved method for fabricating biological cell mass model that eliminates or at least diminishes the disadvantages and problems described above.

### SUMMARY OF INVENTION

Accordingly, a first aspect of the present invention provides a high-throughput selection of target cell masses and fabrication method for biomaterial-encapsulated cell mass (BECM) model under a controlled manner in terms of the number of cell mass, dimension, composition, and number of cells per mass unit with consistency (repeatability). The as-fabricated BECM model is ready for use in a wide range of applications without further cultivation or pre-conditioning.

Exemplarily, the method in the first aspect includes:
providing one or more cell masses in an intact state or partially digested state;
subjecting the intact or partially digested cell masses to a cell imaging device;
providing a formulation of a biomaterial to encapsulate the cell masses in-situ;
acquiring images of the cell masses to target cell masses to be encapsulated into the biomaterial;
determining location, dimension, cell number, cell phenotype, and optical signal intensity of the target cell masses with respect to non-targeted cell masses in the images; and
mixing the formulation of the biomaterial with the target cell masses in the corresponding compartment followed by inducing a formation of a network, gelation system or scaffold for encapsulating and accommodating the target cell masses.

In certain embodiments, the one or more cell masses are sourced from primary, secondary or modified cell lines, or a tissue or biopsy of the same or different subject than a recipient of the BECM or the cells derived therefrom.

In certain embodiments, the one or more cell masses include spheroids and organoids.

In certain embodiments, a precursor of the biomaterial can be any non-cytotoxic and biocompatible material or mixture that forms a network, gelation system, or scaffold for encapsulating and accommodating the one or more cell masses upon stimulation by one or more elements including, but not limited to, light irradiation, temperature change, pH change, and change in chemical composition surrounding the biomaterial.

In certain embodiments, the formulation of the biomaterial comprises a photo-crosslinkable hydrogel as the precursor of the biomaterial.

In certain embodiments, the photo-crosslinkable hydrogel is gelatin-methacryloyl (Gel-MA), alginate-methacryloyl, hyaluronic acid-methacryloyl, fibroin-methacryloyl, chitosan-methacryloyl, poly(ethylene glycol)-methacryloyl, dextran-methacryloyl, poly-lysine-methacryloyl, or F127-methacryloyl, or any combination thereof.

In certain embodiments, the photo-crosslinkable hydrogel can be a mixture of more than one of acrylated polymers, and the ratio between different acrylated polymers in the mixture varies according to the desired viscosity of the mixture and/or strength of the resulting biomaterial after crosslinking.

In certain embodiments, the formulation of the biomaterial further comprises a photo-initiator.

In certain embodiments, the photo-initiator is lithium phenyl-2,4,6-trimethylbenzoylphosphinate (LAP), 2-hydroxy-4′-(2-hydroxyethoxy)-2-methylpropiophenone, or 2,4,6-trimethylbenzoyldi-phenylphosphinate, or any combination thereof.

In certain embodiments, the cell imaging device comprises a microscope, one or more compartments for in-situ biomaterial encapsulation of the cell masses, and a camera.

In certain embodiments, each of the one or more compartments comprises a temperature control mechanism and a gas regulation mechanism to maintain cell cultivation conditions for the cell masses.

In certain embodiments, the microscope can acquire visible light, fluorescent, and luminescent signals from the cell masses, and the camera can output bright-field, dark-field, fluorescent, and luminescent images thereof.

In certain embodiments, the target cell masses identified on the images will be mapped in the corresponding compartment(s) after mixing with the formulation of the biomaterial.

In certain embodiments, the mapping of the target cell masses in the compartment is performed by an image analysis algorithm or manually by a user of the cell imaging device.

In certain embodiments, the image analysis algorithm can differentiate the image signals from the target cell masses than those from the non-target cell masses or any noise from the background, and also calculate distance between the target cell masses and neighboring non-target cell masses.

In certain embodiments, the target cell masses have an average diameter from about 30 to 2000 μm.

In certain embodiments, the target cell masses may have an average diameter from about 80 to 100 μm.

In certain embodiments, the target cell masses emit optical signals detectable by the cell imaging device representing expression of one or more biomarkers by one or more cell phenotypes of interest.

In certain embodiments, each of the target cell masses and neighboring non-target cell masses are separated by a distance from larger than 0 μm to 2000 μm, more specifically, from at least 200 μm to 2000 μm.

In certain embodiments, the non-target cell masses may have an average diameter larger than 20 μm.

In certain embodiments, the formulation of the biomaterial is pre-warmed to 37° C. prior to mixing with the cell masses.

In certain embodiments, the formulation of the biomaterial and the cell masses are mixed in the compartment in a volume ratio of 1:1.

In certain embodiments, the formation of the biomaterial is induced to form the network, gelation system, or scaffold by the stimulation specifically applied to the compartment at where the target cell masses are mapped from the images.

In certain embodiments, the photo-crosslinkable hydrogel in the presence of the photo-initiator is cross-linked under light irradiation to a region in the compartment where the target cell masses are mapped from the images.

In certain embodiments, the formulation of the biomaterial further comprises one or more biomolecules including, but not limited to, one or more microorganisms, growth factors, substrates for facilitating cultivation, maintenance and/or differentiation of the target cell masses.

In certain embodiments, the method further includes sorting the as-fabricated BECM by a sorting mechanism including, but not limited to, a light scattering device.

A second aspect of the present invention provides a biomaterial-encapsulated cell mass model fabricated according to the method described in the first aspect and various embodiments of the present invention.

A third aspect of the present invention provides uses of or methods for using the biomaterial-encapsulated cell mass model or biological cells derived therefrom in different areas including drug screening, pharmacokinetic study, drug resistance study, cancer staging, metastasis study, as a xenograft in in vivo models, as an implant for regenerative medicine, or selection of certain cell phenotypes from a sample, tissue or biopsy, immunotherapy, and a platform for studying molecular pathway, stem cell research, or developmental biology.

In certain embodiments, the biomaterial-encapsulated cell mass model can be configured into a form of three-dimensional structures, motifs, diseased tissue models or droplets by an extrusion-based three-dimensional (3-D) bioprinter comprising a nozzle extrusion mechanism after sorting the desired BECMs or cells derived therefrom by the sorting mechanism from a pool of as-fabricated BECMs in terms of their size, composition of cells, cell number, phenotypes, and/or light signal intensity, etc., in order to enable a high-throughput BECM-based analysis.

This summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. Other aspects of the present invention are disclosed as illustrated by the embodiments hereinafter.

### BRIEF DESCRIPTION OF DRAWINGS

The patent or application file contains at least one drawing executed in color. Copies of this patent or patent application publication with color drawing(s) will be provided by the Office upon request and payment of the necessary fec.

The appended drawings, where like reference numerals refer to identical or functionally similar elements, contain figures of certain embodiments to further illustrate and clarify the above and other aspects, advantages and features of the present invention. It will be appreciated that these drawings depict embodiments of the invention and are not intended to limit its scope. The invention will be described and explained with additional specificity and detail through the use of the accompanying drawings in which:

FIG. 1 shows microscope images of HT-29 spheroids grown on a low attachment culture dish;

FIG. 2 shows bright-field and fluorescent images of micrometer-sized spheroids spread in a GelMA-based gelation system in a compartment of a cell imaging device according to certain embodiments: (a) green, (b) red, and (c) merged fluorescent images of cells and spheroids; (d) a merged brightfield and fluorescence image;

FIG. 3 shows image analysis results for determining positions, size and composition of selected spheroids according to certain embodiments: (a) merged fluorescence images generated from the camera; (b) the area with colocalized green and red fluorescence signals; (c) the image analysis algorithm-generated binary image showing the position of the spheroids selected from (b) with desired size and composition;

FIG. 4 shows a top view of compartments containing spheroids and biomaterial for encapsulation of selected spheroids according to certain embodiments, where the circular spots denote the irradiation area of 400 μm and the center of each spot is the selected spheroid;

FIG. 5A shows bright-field images of biomaterial-encapsulated spheroids fabricated according to certain embodiments;

FIG. 5B shows an enlarged view of randomly selected biomaterial-encapsulated spheroids from the bright-field images of FIG. 5A;

FIG. 6 shows live/dead cell staining of a biomaterial-encapsulated spheroid 3 days after encapsulation according to certain embodiments: (a) propidium iodide; (b) calcein AM; (c) the merged fluorescence images of (a) and (b); (d) bright-field image;

FIG. 7 shows a dose-response curve of biomaterial-encapsulated HT-29 cancer cell spheroids treated with Osimertinib for 72 h (n=10);

FIG. 8 shows selected HT-29 spheroids with a narrow size range after releasing from gelation system of the biomaterial: scale bars represent 100 μm;

FIG. 9A shows an image from a top view of high-throughput formation of HT-29 cancer cell spheroids encapsulated in an epithelial cell laden cancer model in a 96-well plate by printing according to certain embodiments;

FIG. 9B shows an image from a top view of high-throughput formation of HT-29 cancer cell spheroids encapsulated in an epithelial cell laden cancer model in a 384-well plate by printing according to certain embodiments;

FIG. 10 shows images of two centimeter-sized HT-29 cancer cell spheroids encapsulated in an epithelial cell laden cancer model formed in a 6-well plate;

FIG. 11 is a flow chart showing steps of selective bio-fabrication of BECMs and sorting of as-fabricated BECMs for subsequent high-throughput analysis according to certain embodiments of the present invention.

Skilled artisans will appreciate that elements in the figures are illustrated for simplicity and clarity and have not necessarily been depicted to scale.

### DETAILED DESCRIPTION OF THE INVENTION

It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

The follow examples are provided to assist the understanding of enabling the present invention and should not be considered limiting the scope of the present invention. The scope of the present invention should be referred to the appended claims.

### Example 1—Fabrication of Colon Cancer Spheroid Model From Colorectal Adenocarcinoma Cell Line HT-29

### 1.1. Spheroid Culturing:

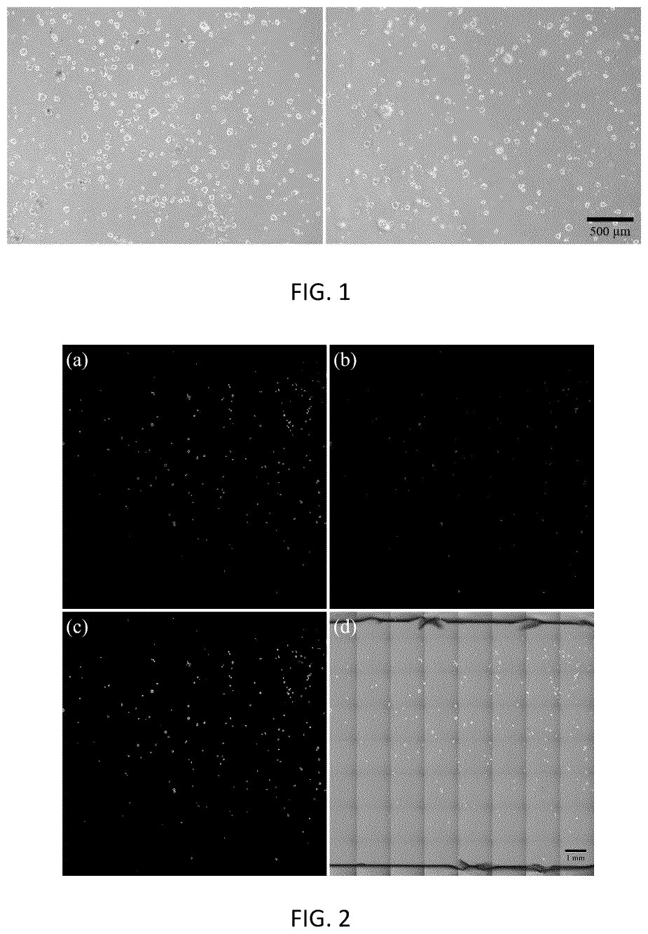

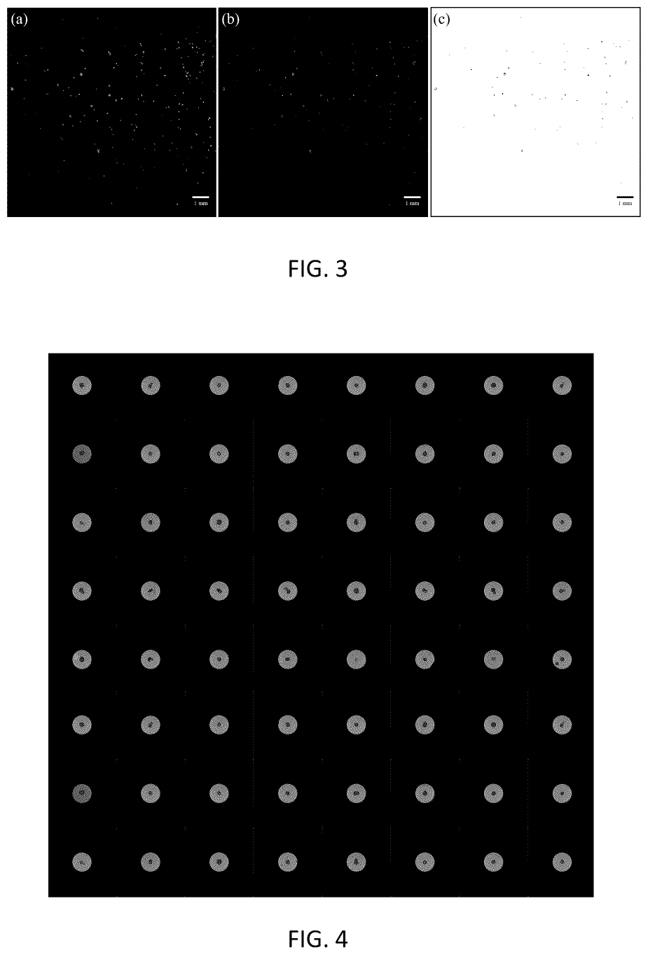

Spheroids of colorectal adenocarcinoma HT-29 were grown on a low-attachment culture dish and were maintained in RPMI-1640 medium supplemented with 10% fetal bovine serum and penicillin-streptomycin. All cells were grown at 37° C. in a humidified 5% CO2 atmosphere before harvesting. Spheroids were formed from micron- to millimeter-size in their average diameter. An example of the spheroids formed as such is shown in FIG. 1.

### 1.2. Preparation of Biomaterial:

Gelatin-methacryloyl (GelMA) based photo-crosslinkable hydrogel was used as the biomaterial for encapsulation in this example.

Formulation of the GelMA based photo-crosslinkable hydrogel included gelatin-methacryloyl with 90 bloom and a photoinitiator, lithium phenyl-2,4,6-trimethylbenzoylphosphinate (LAP). Initially, 20% v/v of GelMA and 0.5% v/v LAP were dissolved in phosphate buffered saline (PBS) as stock solution. Before use, the formulation was pre-warmed to 37° C.

### 1.3. Preparation of Fluorescence-Labeled Spheroids:

The spheroids collected from the culture dish were filtered through a mesh with specific cut-off size substantially smaller than the target spheroids but larger than other masses than the target spheroids, e.g., in a size range from 100 to 5000 μm. They were then stained with specific fluorescence probes to colon carcinoma, e.g., Calcein AM. Then GelMA was mixed with the spheroid suspension in a volume ratio of 1:1. The biomaterial was loaded into a chamber containing multiple compartments. The fluorescence-labeled spheroids of various sizes were spread and ready for analysis.

### 1.4. Imaging and Image Analysis:

An imaging system containing a fluorescent microscope was used to scan the whole chamber of cells using visible light, and lasers with wavelengths of 488 nm and 633 nm. Bright-field and fluorescent images of the whole chamber were captured.

The captured images by the camera of the microscope were imported to an image analysis software to analyze the following parameters of each spheroid including size, fluorescent signal of each channel, surface uniformity, the distance between other spheroids or debris, etc. Particular values of the above parameters were input as selection criteria and the software would select the spheroids according to the values of interest. The spheroids ranging from 80-100 μm in diameter, with both green and red fluorescent signals inside the spheroid, and with at least 200 μm of separation between the target spheroid and other neighboring, non-target cell masses which exceeded 20 μm in diameter were selected. The software then generated three-dimensional (3-D) coordinates of all the selected spheroids and proceeded to GelMA encapsulation.

### 1.5. Biomaterial Encapsulation

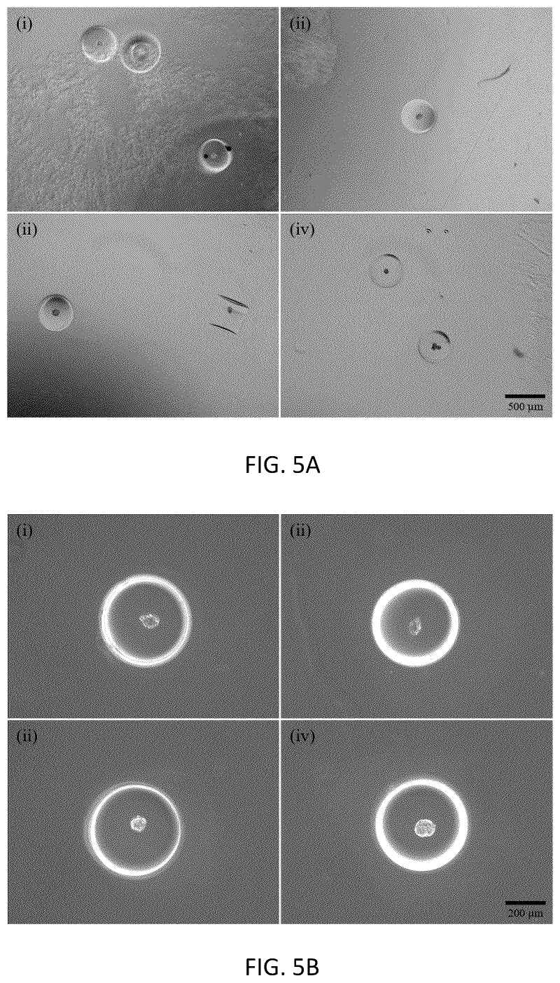

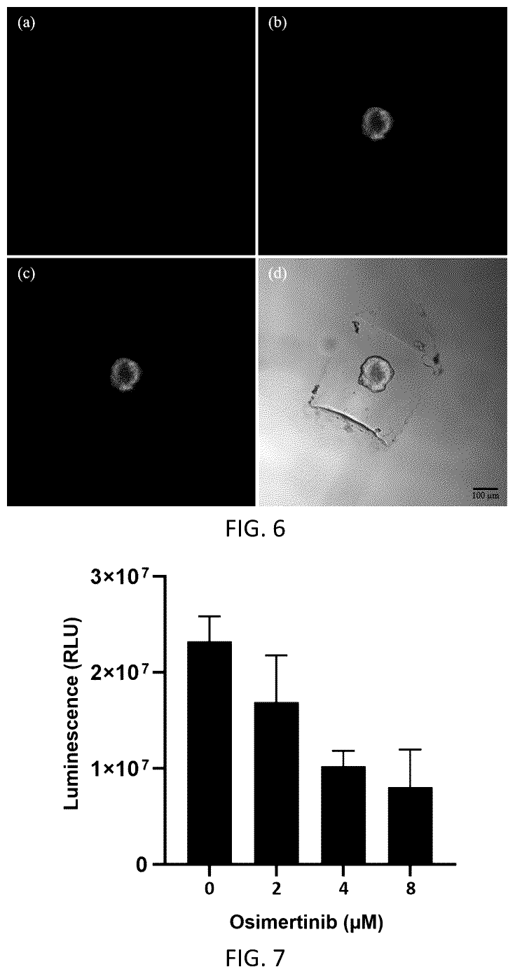

Light irradiation of the coordinates of interest to crosslink the GelMA around the target cell masses. A gel size of 400 μm in diameter was used to enclose each of the target spheroids. 100 seconds of light irradiation with a wavelength of 405 nm was performed to complete the cross-linking process, generating gel-encapsulated spheroids where each spheroid had a diameter of about 80-100 μm and both green and red fluorescent signals inside the spheroids (FIGS. 4-6).

### 1.6. BECM Filtering and Collection:

After BECM formation, warm PBS was used to wash away floating cells, non-target spheroids and debris. The remaining BECMs were filtered, collected, and optionally sorted into each well of a 96-well plate (or 384-well plate, or other multi-well plate) for subsequent use.

### Example 2—Drug Response of BECM to Cancer Therapeutics

Taking the HT-29 spheroids encapsulated BECM fabricated in Example 1, each of the BECMs was sorted into each well of a 96-well plate, and incubated with an FDA-approved, EGFR blocker for non-small cell lung cancer (NSCLC) called Osimertinib in various concentrations (0, 2, 4, and 8 μM) for 72 hrs at 37° C. in a humidified 5% CO2 atmosphere. Commercially available 3D cell viability assay, CELLTITER GLO (Promega Corp.), was used to determine the cell viability of the BECMs. The results from FIG. 7 show that a typical 3D cell culture viability dose-response was obtained, similar to that of the other established NSCLC in vitro cell models.

### Example 3—High-Throughput Fabrication of Cell Models Based on Spheroids or Organoids Isolated from BECMs

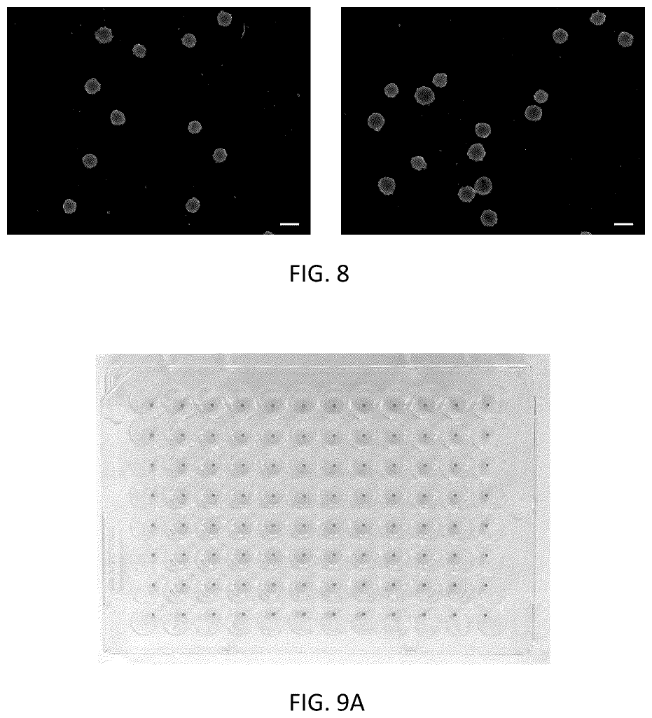

Initially, HT-29 cell model was generated following the method described in Example 1. After formation of BECMs, warm PBS was used to wash away floating cells, other non-target spheroids and debris before filtering and collecting the BECMs. The collected BECMs were treated with 1× collagenase for 10 min in PBS to release the encapsulated spheroids from the gelation system to obtain highly regular spheroids (FIG. 8). The highly regular spheroids were then mixed with an epithelial-cell-laden hydrogel that comprised 1×10⁷ cell mL⁻¹ FHC cell, 3% sodium alginate, and 9% methylcellulose in a 1:4 ratio (spheroid:hydrogel, v/v).

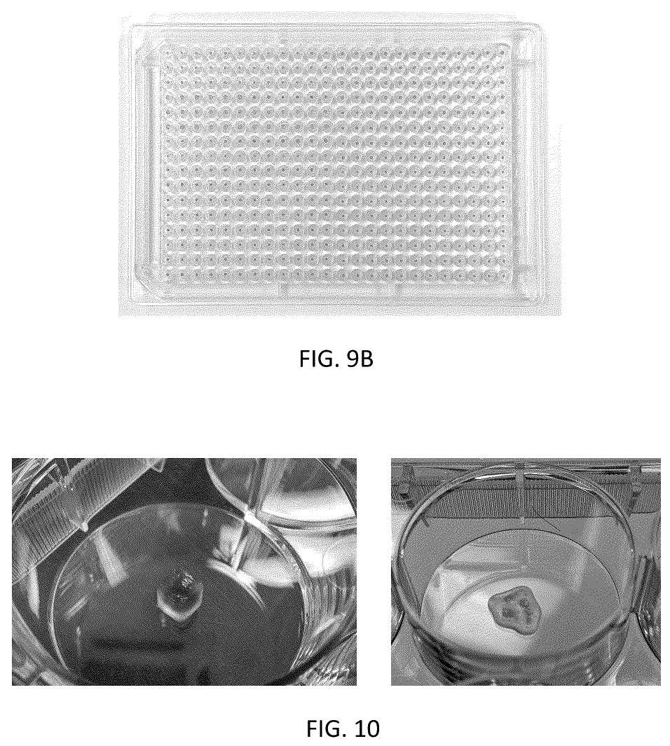

A colon cancer tissue model was chosen from the software in an extrusion-based 3-D bioprinter, or abbreviated as MAPS. The model was printed in a 96-well plate (FIG. 9A) and a 384-well plate (FIG. 9B). The structures were crosslinked by using 25 mM CaCl₂) in normal saline for 30 min, followed by washing with PBS and replenishing with complete RPMI using the robotic liquid handler in MAPS.

Alternatively, two other colon cancer tissue models were chosen from the software in MAPS. The models were printed in a 6-well plate (FIG. 10). The structures were crosslinked by using 25 mM CaCl₂) in normal saline for 30 min, followed by washing with PBS and replenishing with complete RPMI using the robotic liquid handler in MAPS.

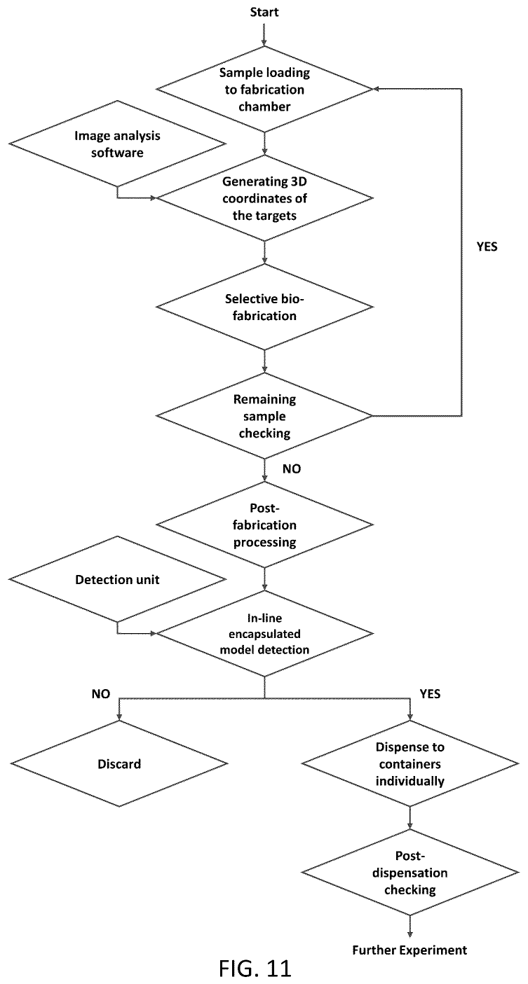

FIG. 11 summarizes the key steps of the present method from sample loading to a bio-fabrication chamber containing multiple compartments, analysis of the images of the loaded sample containing both the target and non-target cell masses in the chamber acquired by the cell imaging device, generation of 3-D coordinates of target cell masses for selective bio-fabrication by a corresponding image analysis algorithm, induction of biomaterial encapsulation after selection, and a post bio-fabrication checking and processing. For subsequent high-throughput analysis based on the as-fabricated BECMs, FIG. 11 further shows the key steps of sorting the as-fabricated BECMs according to certain requirements for said subsequent analysis including dispensation of the selected BECMs into a designated container (e.g., 96- or 384-well plate) for high-throughput analysis and a post-dispensation checking before the analysis starts.

Although the invention has been described in terms of certain embodiments, other embodiments apparent to those of ordinary skill in the art are also within the scope of this invention. Accordingly, the scope of the invention is intended to be defined only by the claims which follow.

1. A method for selecting cell masses from a source and fabricating a biomaterial-encapsulated cell mass model (BECM) based on the selected cell masses, the method comprising:
providing one or more cell masses in an intact or partially digested state;
subjecting the intact or partially digested cell masses to a cell imaging device;
providing a formulation of a biomaterial to encapsulate the cell masses in-situ;
acquiring images of the cell masses to target cell masses to be encapsulated into the biomaterial;
determining location, dimension, cell number, cell phenotype, and optical signal intensity of the target cell masses with respect to non-targeted cell masses in the images; and
mixing the formulation of the biomaterial with the target cell masses in the corresponding compartment followed by inducing a formation of a network, gelation system or scaffold for encapsulating and accommodating the target cell masses.

2. The method of claim 1, wherein the one or more cell masses are sourced from primary, secondary or modified cell lines, or a tissue or biopsy of the same or different subject than a recipient of the BECM or the cells derived therefrom.

3. The method of claim 1, wherein the one or more cell masses include spheroids and organoids.

4. The method of claim 1, wherein the formulation comprises a precursor of the biomaterial which is a non-cytotoxic and biocompatible material or mixture that forms a network, gelation system, or scaffold for encapsulating and accommodating the target cell masses upon stimulation by one or more elements comprising light irradiation, temperature change, pH change, and change in chemical composition surrounding the biomaterial.

5. The method of claim 4, wherein the precursor is a photo-crosslinkable hydrogel.

6. The method of claim 5, wherein the photo-crosslinkable hydrogel is gelatin-methacryloyl (Gel-MA), alginate-methacryloyl, hyaluronic acid-methacryloyl, fibroin-methacryloyl, chitosan-methacryloyl, poly(ethylene glycol)-methacryloyl, dextran-methacryloyl, poly-lysine-methacryloyl, or F127-methacryloyl, or any combination thereof.

7. The method of claim 1, wherein the formulation of the biomaterial further comprises a photo-initiator.

8. The method of claim 7, wherein the photo-initiator is lithium phenyl-2,4,6-trimethylbenzoylphosphinate (LAP), 2-hydroxy-4′-(2-hydroxyethoxy)-2-methylpropiophenone, or 2,4,6-trimethylbenzoyldi-phenylphosphinate, or any combination thereof.

9. The method of claim 1, wherein the formulation of the biomaterial and the target cell masses are mixed in the compartment in a volume ratio of 1:1.

10. The method of claim 1, wherein the cell imaging device comprises a microscope for acquiring visible light, fluorescent, and luminescent signals from the cell masses, one or more compartments comprising a temperature control mechanism and a gas regulation mechanism for in-situ biomaterial encapsulation and maintaining cell cultivation conditions of the target cell masses, and a camera for outputting images of the cell masses comprising bright-field, dark-field, fluorescent, and luminescent images.

11. (canceled)

12. (canceled)

13. The method of claim 10, further comprising mapping the target cell masses determined on the images in the corresponding compartment(s) after said mixing the formulation of the biomaterial with the target cell masses.

14. The method of claim 13, wherein said mapping the target cell masses determined on the images in the compartment is performed by an image analysis algorithm or manually by a user of the cell imaging device.

15. The method of claim 14, wherein the image analysis algorithm is configured to differentiate the image signals from the target cell masses than those from the non-target cell masses or any noise from the background, and also calculate distance between the target cell masses and neighboring non-target cell masses.

16. The method of claim 1, wherein the target cell masses have an average diameter from about 30 to 2000 μm.

17. The method of claim 1, wherein the target cell masses emit optical signals detectable by the cell imaging device representing expression of one or more biomarkers by one or more cell phenotypes of interest.

18. The method of claim 15, wherein each of the target cell masses and neighboring non-target cell masses are separated by a distance from larger than 0 μm to 2000 μm.

19. The method of claim 13, wherein a stimulation to induce the precursor of the biomaterial to form the network, gelation system, or scaffold for encapsulating and accommodating the target cell masses is specifically applied to the compartment at where the target cell masses are mapped from the images.

20. The method of claim 19, wherein the precursor in the presence of the photo-initiator is cross-linked under a light irradiation to a region in the compartment where the target cell masses are mapped from the images.

21. (canceled)

22. The method of claim 19, further comprising sorting the fabricated BECM by a sorting mechanism comprising a light scattering device.

23. A biomaterial-encapsulated cell mass model fabricated according to the method of claim 1.

24. (canceled)

25. (canceled)
